(19) United States
(12) Patent Application Publication
(10) Pub. No.: US 2019/0221052 A1
(43) Pub. Date: Jul. 18, 2019

(54) VEHICLE OPERATION MANAGEMENT SYSTEM

(71) Applicant: DENSO CORPORATION, Kariya-city (JP)

(72) Inventors: Daisuke KITAZAWA, Kariya-city (JP); Keiji OKAMOTO, Kariya-city (JP); Kimiaki TANAKA, Kariya-city (JP)

(21) Appl. No.: 16/364,227

(22) Filed: Mar. 26, 2019

Related U.S. Application Data
(63) Continuation of application No. PCT/JP2017/028050, filed on Aug. 2, 2017.

(30) Foreign Application Priority Data
Sep. 29, 2016 (JP) 2016-191270

(51) Int. Cl. G07C 5/00; G05D 1/00; B60K 28/06; G08G 1/0962; A61B 5/18

(52) U.S. Cl. CPC G07C 5/008; G05D 1/0027; G05D 1/0055; G05D 2201/0213; G08G 1/0962; A61B 5/18; B60K 28/06

(57) ABSTRACT

A vehicle operation management system includes: multiple vehicle management devices that are respectively mounted on vehicles; and a center management device that is disposed at an operation management center and is communicable with the vehicle management devices. The vehicle management device includes: a vehicle information acquisition section; an image acquisition section; a driver's condition determination section; and an information transmitter. The center management device includes: an information receiver; an information display section; and a speech section.

### CROSS REFERENCE TO RELATED APPLICATIONS

The present application is a continuation application of International Patent Application No. PCT/JP2017/028050 filed on Aug. 2, 2017, which designated the United States and claims the benefit of priority from Japanese Patent Application No. 2016-191270 filed on Sep. 29, 2016. The entire disclosures of all of the above applications are incorporated herein by reference.

### TECHNICAL FIELD

The present disclosure relates to a vehicle operation management system.

### BACKGROUND

A related art describes a configuration for transmission of a detection result from an onboard device to an operation management server at an operation management center, and determination at the operation management server of whether the driver is in a condition appropriate for driving.

### SUMMARY

The present disclosure according to one aspect provides a vehicle operation management system which includes multiple vehicle management devices that are respectively mounted on vehicles, and a center management device that is disposed at an operation management center and is communicable with the vehicle management devices. Each of the vehicle management devices acquires vehicle information and a driver image; and determines whether a driver's condition is abnormal; and transmits the vehicle information and video information. The center management device receives the vehicle information and the video information; displays the vehicle information and the video information; and allows an operator to communicate with a driver.

### BRIEF DESCRIPTION OF DRAWINGS

The above and other objects, features, and advantages of the present disclosure will become more apparent from the following detailed description with reference to the accompanying drawings. In the drawings:

FIG. 1 is a functional block diagram depicting a schematic entire configuration of a vehicle operation management system according to a first embodiment;

FIG. 2 is an explanatory flowchart of control executed by the vehicle operation management system;

FIG. 3 is an explanatory flowchart of control executed by a vehicle operation management system according to a second embodiment; and

FIG. 4 is an explanatory flowchart of control executed by a vehicle operation management system according to a third embodiment.

### DETAILED DESCRIPTION

A related art describes a configuration for transmission of a detection result of alcohol concentration in driver's breath and a detection result of driver's blood pressure from an onboard device to an operation management server at an operation management center, and determination at the operation management server of whether the driver is in a condition appropriate for driving. According to this configuration, it may be possible to prevent accidents by searching for a substitute driver to be replaced with the driver when the driver is determined as being in a condition inappropriate for driving.

The above configuration may include determination of whether the driver is in a condition appropriate for driving according to only biological information like the alcohol concentration in driver's breath and the driver's blood pressure. The driver's condition may be not determined and managed by an operator at the operation management center on a real-time basis.

The present disclosure may provide a vehicle operation management system that allows an operator at an operation management center to determine abnormality in driver's condition on a real-time basis.

A vehicle operation management system according to a first aspect of the present disclosure may include multiple vehicle management devices that are respectively mounted on vehicles, and a center management device that is disposed at an operation management center and is communicable with the vehicle management devices. Each of the vehicle management devices may include: a vehicle information acquisition section that acquires vehicle information; an image acquisition section that acquires a driver image; a driver's condition determination section that determines whether a driver's condition is abnormal; and an information transmitter that transmits the vehicle information and low-resolution video information on the driver image to the center management device at normal time, and transmits the vehicle information and high-resolution video information on the driver image to the center management device when the driver's condition is abnormal. The center management device may include: an information receiver that receives the vehicle information and the low-resolution video information on the driver image or the high-resolution video information on the driver image from each of the vehicle management devices, an information display section that displays the vehicle information and the video information on the driver image; and a speech section that allows an operator at the operation management center to communicate with a driver of the vehicles via the vehicle management devices. The information display section of the center management device may display video information having resolution unified to a set value for display of the video information on the driver image when the driver's condition is abnormal.

### First Embodiment

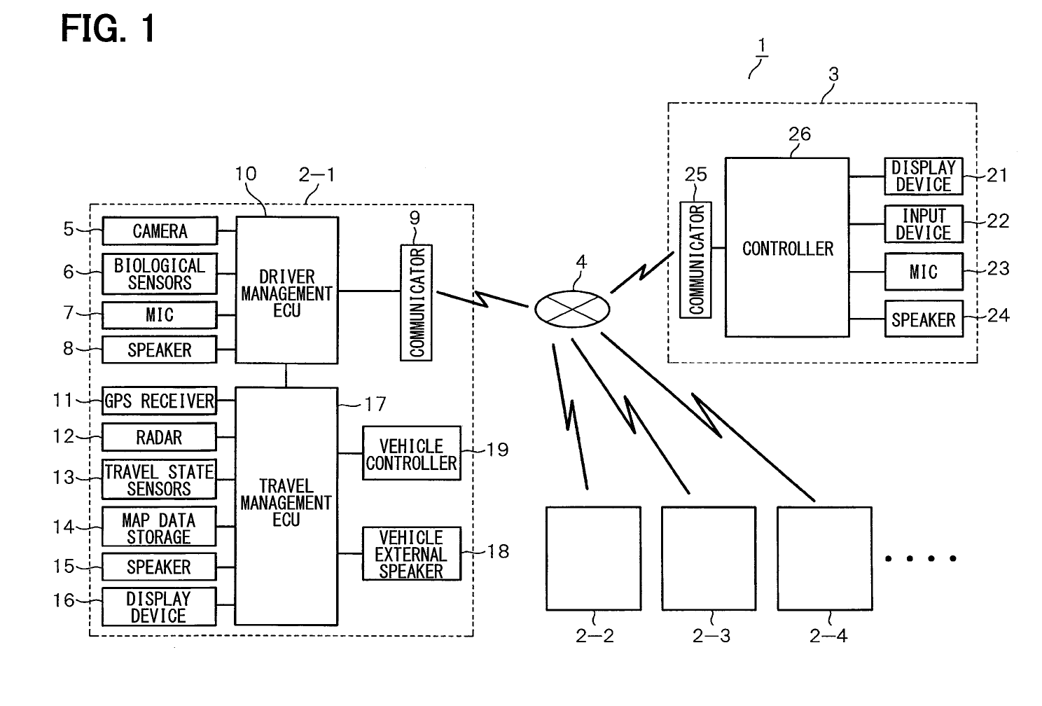

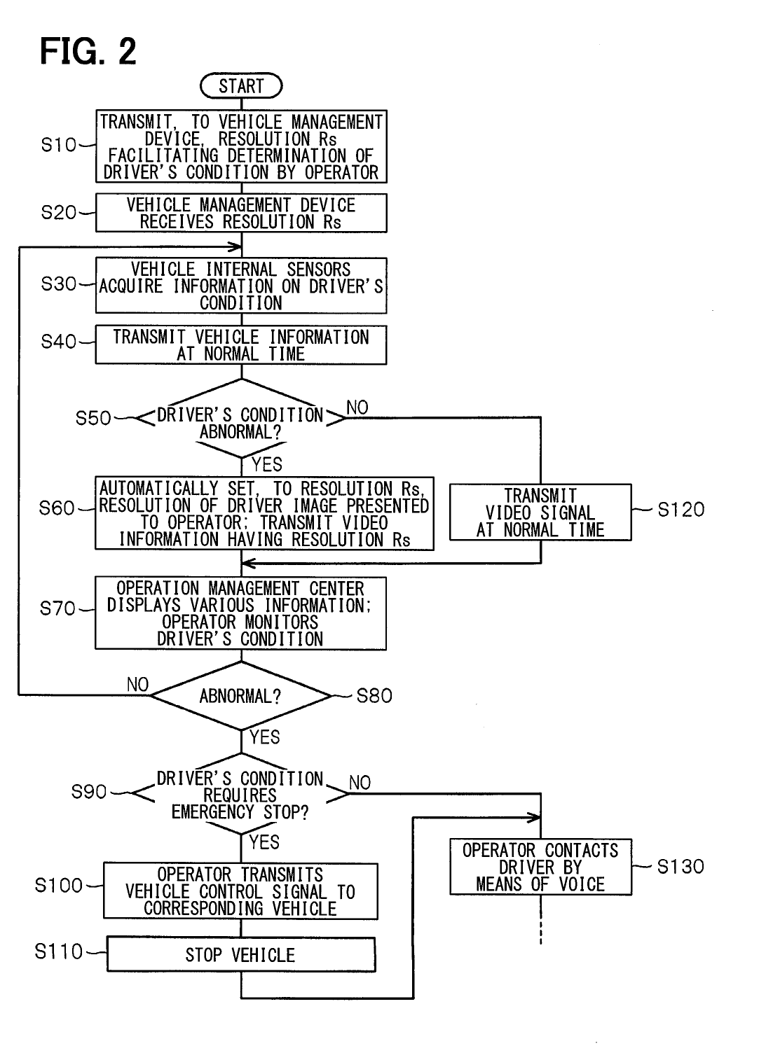

The first embodiment will be described below with reference to FIG. 1 and FIG. 2. As shown in FIG. 1, the present embodiment provides a vehicle operation management system 1 that is configured to manage operation of at least one vehicle, and includes vehicle management devices 2-1, 2-2, 2-3, 2-4 or the like mounted on respective vehicles, and a center management device 3 disposed at an operation management center. The vehicle management devices 2-1, 2-2, 2-3, 2-4 or the like and the center management device 3 are communicably connected to each other via a wireless communication network serving as a public network like a mobile phone network 4.

The vehicle management devices 2-1, 2-2, 2-3, 2-4 or the like are configured substantially identically, so that the vehicle management device 2-1 will be described in terms of a specific configuration thereof. The vehicle management device 2-1 includes a camera 5, a biological sensors 6, a microphone 7, a speaker 8, a communicator 9, a driver management ECU 10, a GPS receiver 11, a radar 12, travel state sensors 13, a map data storage 14, a speaker 15, a display device 16, a travel management ECU 17, a vehicle external speaker 18, and a vehicle controller 19. ECU is a kind of abbreviation for electronic control unit.

The camera 5 captures driving posture, a face or the like of a driver, and outputs the captured driver image to the driver management ECU 10. The biological sensors 6 include various types of sensors that detect pulses, blood pressure, brain waves, an electrocardiogram, and the like of the driver, and output such biological information on the driver thus detected to the driver management ECU 10. The microphone 7 receives voice or the like generated by the driver, converts the voice into a voice signal, and outputs the voice signal thus obtained to the driver management ECU 10. The speaker 8 receives a sound signal of voice, a music piece, or the like output from the driver management ECU 10, and outputs sound of the voice, the music piece, or the like.

The communicator 9 has a function to execute wide-area communication connected to the public network like the mobile phone network 4, to communicate with the center management device 3 at the operation management center via the mobile phone network 4 for transmission, reception, or the like of communication or data.

The driver management ECU 10 has functions to receive (i.e. acquire) the driver image from the camera 5 and the biological information on the driver from the biological sensors 6, determine a driver's condition in accordance with the information thus received, and determine whether the driver has difficulty in vehicle driving in accordance with a determination result of the driver's condition. At normal time when the driver is determined as having no difficulty in vehicle driving, the driver management ECU 10 is configured to transmit low-resolution video information on the driver image (e.g. information of about three hundred thousand pixels) and vehicle information to the center management device 3 at set time intervals (i.e. periodically). The normal time may correspond to a case when the driver's condition is normal, or a case when the driver is in a normal condition. For transmission of the low-resolution video information on the driver image, the vehicle management devices 2-1, 2-2, 2-3, 2-4 or the like mounted on the plurality of vehicles are configured to transmit video information having substantially equal resolution.

At abnormal time when the driver is determined as having difficulty in vehicle driving, the driver management ECU 10 transmits, to the center management device 3 via the communicator 9, information indicating that the driver has difficulty in vehicle driving, information on the determination result of the driver's condition, high-resolution video information on the driver image (i.e. video information having resolution higher than the resolution of the video information transmitted at normal time), the biological information on the driver, vehicle information acquired as to be described later, and the like.

For transmission of the high-resolution video information on the driver image, the vehicle management devices 2-1, 2-2, 2-3, 2-4 or the like mounted on the plurality of vehicles are configured to transmit video information widely varied in resolution among the vehicles (e.g. video information of about three to ten million pixels). In other words, the vehicle management devices 2-1, 2-2, 2-3, 2-4 or the like are varied in product specification (e.g. resolution specification of video information) depending on manufacturers and years of manufacture, and devices manufactured earlier typically tend to have lower resolution specification.

The driver management ECU 10 in each of the vehicle management devices 2-1, 2-2, 2-3, 2-4 or the like according to the present embodiment is configured to receive, from the center management device 3 at the operation management center, to store resolution Rs (e.g. about three million pixels) of the video information on the driver image transmitted at abnormal time, and transmit, to the center management device 3, video information on the driver image converted to have the resolution Rs thus stored. The resolution Rs corresponds to resolution unified to a set value. The driver management ECU 10 in each of the vehicle management devices 2-1, 2-2, 2-3, 2-4 or the like, which are varied in product specification (e.g. resolution specification of video information) depending on manufacturers and years of manufacture, may possibly transmit a video signal having different resolution in transmitting, to the center management device 3, the high-resolution video information on the driver image at abnormal time. In contrast, the present embodiment includes unifying to the resolution Rs, so that the driver management ECU 10 in each of the vehicle management devices 2-1, 2-2, 2-3, 2-4 or the like is configured to transmit, to the center management device 3, video information having the unified high resolution.

Information obtained by combining the low-resolution video information at normal time and the vehicle information is much smaller in data volume than information obtained by combining the various information transmitted when the driver has difficulty in driving. In this case, the low-resolution video information and the like are transmitted at communication speed of a communication line (i.e. the mobile phone network 4) being set to a low level.

The driver management ECU 10 is configured to allow the driver to communicate, by using the microphone 7 and the speaker 8, with an operator operating the center management device 3 at the operation management center via the communicator 9 and the mobile phone network 4. When receiving a stop command signal for vehicle stop, from the center management device 3 (i.e. the operator) at the operation management center, the driver management ECU 10 is configured to transmit the stop command signal to the travel management ECU 17 for vehicle stop.

The driver management ECU 10 has functions as a vehicle information acquisition section, an image acquisition section, a biological information acquisition section, a driver's condition determination section, an information transmitter, and a voice information acquisition section.

The GPS receiver 11 detects a current position (e.g. latitude/longitude information) of the vehicle in accordance with a received GPS signal, and transmits information on the detected current position to the travel management ECU 17. The radar 12 detects an obstacle or the like located ahead of the vehicle, and transmits information on the detected obstacle (e.g. distance information) to the travel management ECU 17. The travel state sensors 13 include various sensors, ECUs, or the like that detect vehicle information indicating a current state of the vehicle (e.g. the current position, travelable distance, an engine state, states of various vehicle sensors, a tire state, an automatically drivable state, vehicle speed, an accelerator position, a brake manipulation amount, acceleration, a steering manipulation amount, lighting states of various lights, or a lighting state of an alarm lamp), and transmits the vehicle information thus detected to the travel management ECU 17.

The map data storage 14 includes a semiconductor memory, a hard disk, a DVD, or the like, and stores map data to be referred to for achievement of a navigation function (e.g. road map data and speed limit data, topography data, facility data, map matching data, route guiding voice data, or the like all over Japan). The travel management ECU 17 is configured to read the map data stored in the map data storage 14. The travel management ECU 17 outputs, via the speaker 15, voice, alarm sound, or the like expressing various messages, and causes the display device 16 to display a map, navigation, the current position of the vehicle, the various messages, or the like. Examples of the display device 16 include a display device provided at an instrument panel. The travel management ECU 17 is further configured to output voice, alarm sound, or the like expressing the various messages to outside the vehicle via the vehicle external speaker 18.

The travel management ECU 17 has a function to calculate a guided route from the current position to a destination and explain the guided route thus calculated (i.e. a navigation function) when receiving destination information and an automatic drive signal from the driver management ECU 10. The travel management ECU 17 can alternatively be configured to drive-control an accelerator, a brake, and the like of the vehicle via the vehicle controller 19 to stop the vehicle. The travel management ECU 17 thus configured functions as a vehicle stop section. The travel management ECU 17 is also configured to transmit, to the driver management ECU 10, the information on the current position, the obstacle information, the vehicle information, the map data, and the like received from the GPS receiver 11, the radar 12, the travel state sensors 13, and the map data storage 14.

The center management device 3 at the operation management center includes a display device 21, an input device 22, a microphone 23, a speaker 24, a communicator 25, and a controller 26. The display device 21 is configured by a liquid crystal display, an organic EL display, or the like that achieves colored presentation. The input device 22 includes a keyboard, a mouse, and the like. The microphone 23 and the speaker 24 receive utterance voice and output received voice during communication by the operator at the operation management center.

The communicator 25 has a function to execute wide-area communication connected to the public network like the mobile phone network 4, to communicate with the vehicle management devices 2-1, 2-2, 2-3, 2-4 or the like, a different information terminal, or the like via the mobile phone network 4 for transmission, reception, or the like of communication or data.

At normal time when the driver has no difficulty in vehicle driving, the controller 26 periodically receives low-resolution video information on the driver image and vehicle information from the vehicle management devices 2-1, 2-2, 2-3, 2-4 or the like, and causes the display device 21 to display the various information thus received. The operator at the operation management center visually recognizes the various information displayed on the display device 21 to monitor whether each driver is driving the vehicle normally.

When any one of the vehicle management devices 2-1, 2-2, 2-3, 2-4 or the like detects that the driver has difficulty in vehicle driving, the controller 26 receives, from the vehicle management devices 2-1, 2-2, 2-3, 2-4 or the like, information indicating that the driver has difficulty in vehicle driving, information on the determination result of the driver's condition, high-resolution video information on the driver image (e.g. information having the resolution Rs), the biological information on the driver, the information on the current position of the vehicle, the vehicle information, and the like, and causes the display device 21 to display the various information thus received. In this case, the driver management ECU 10 in each of the vehicle management devices 2-1, 2-2, 2-3, 2-4 or the like, which are varied in product specification depending on manufacturers and years of manufacture, may possibly transmit a video signal having different resolution in transmitting, to the center management device 3, the high-resolution video information on the driver image at abnormal time. In contrast, the present embodiment includes unifying to the resolution Rs, so that the driver management ECU 10 in each of the vehicle management devices 2-1, 2-2, 2-3, 2-4 or the like is configured to transmit, to the center management device 3, video information having the unified high resolution. The display device 21 accordingly displays the driver images having high resolution unified to the resolution Rs, to allow the operator at the operation management center to visually recognize the driver images of the respective vehicles being equal in resolution and being easy to check.

The operator at the operation management center visually recognizes the various information displayed on the display device 21 to easily find occurrence of abnormality that any driver cannot drive a vehicle, and can communicate with the driver by using the microphone 23 and the speaker 24 via the communicator 25, the mobile phone network 4, and the vehicle management device 2. The operator can thus determine a driver's abnormal condition on a real-time basis (i.e. quickly). The controller 26 thus configured has functions as an information receiver, an information display section, and a speech section.

The operator further operates the input device 22 of the center management device 3 when determining that it is better to stop the vehicle, so that the controller 26 transmits a stop command signal to corresponding one of the vehicle management devices 2-1, 2-2, 2-3, 2-4 or the like to stop the vehicle. The controller 26 functions as a stop command transmitter in this case. When determining that the driver has fainted or the like and needs to be rescued immediately, the operator can still further report to a fire station to arrange dispatch of an ambulance or report to a medical institution, a police station, or the like.

The vehicle management devices 2-1, 2-2, 2-3, 2-4 or the like and the center management device 3 will be described next in terms of control operation thereof with reference to FIG. 2. The vehicle management device 2-1 representing the vehicle management devices 2-1, 2-2, 2-3, 2-4 or the like will be described herein in terms of operation thereof. The remaining vehicle management devices 2-2, 2-3, 2-4 or the like operate substantially similarly.

Initially in step S10 in FIG. 2, the operator at the operation management center operates the center management device 3 to transmit, to the vehicle management device 2-1, the resolution Rs of video information on the driver image to be received at abnormal time. The resolution Rs has a level necessary for display on the display device 21 of a clear driver image facilitating determination of the driver's condition by the operator, and information on the resolution Rs includes various information such as the number of pixels (e.g. about three million pixels), white balance, contrast, and sharpness.

In subsequent step S20, the vehicle management device 2-1 receives the information on the resolution Rs from the center management device 3, and the driver management ECU 10 stores the resolution Rs thus received in an internal memory. In next step S30, the driver management ECU 10 in the vehicle management device 2-1 acquires information on the driver's condition, specifically, video information on the driver image from the camera 5, biological information such as brain waves, an electrocardiogram, and blood pressure of the driver from the biological sensors 6, and the vehicle information (e.g. information on the current position of the vehicle from the GPS receiver 11, or vehicle information from the travel state sensors 13). Subsequently in step S40, the driver management ECU 10 in the vehicle management device 2 transmits, to the center management device 3, the vehicle information thus acquired as information to be transmitted at normal time.

In next step S50, the driver management ECU 10 in the vehicle management device 2-1 determines whether the driver's condition is abnormal in accordance with the information on the driver's condition acquired in step S30. The driver management ECU 10 determines that the driver is in an abnormal condition of being unable to drive a vehicle if the driver is almost asleep during driving, is unconscious, or the like. If the driver's condition is not abnormal (NO) in step S50, the process flow proceeds to step S120 and the driver management ECU 10 in the vehicle management device 2-1 transmits, to the center management device 3, the low-resolution video information (e.g. video information of about three hundred thousand pixels) on the driver image thus acquired, as information to be transmitted at normal time. In this case, the low-resolution video information is transmitted at communication speed of the communication line (i.e. the mobile phone network 4) being set to a low level.

If the driver's condition is abnormal (YES) in step S50, the process flow proceeds to step S60. In step S60, the driver management ECU 10 in the vehicle management device 2-1 sets, to the resolution Rs stored in the memory, high resolution of the driver image to be presented to the operator at abnormal time, in other words, to be transmitted to the center management device 3. In step S60, the driver management ECU 10 transmits, to the center management device 3, as information to be transmitted at abnormal time, information indicating that the driver's condition is abnormal, the video information having the resolution Rs on the driver image thus acquired (i.e. video information having resolution higher than that of the video information to be transmitted at normal time, to have high resolution unified to the set value, such as video information having about three million pixels). In this case, the high-resolution video information and the like are transmitted at communication speed of the communication line (i.e. the mobile phone network 4) being switched to a high level. The driver management ECU 10 according to the present embodiment is configured to transmit in step S60, to the travel management ECU 17, information indicating that the driver's condition is abnormal. The travel management ECU 17 is configured to cause the display device 16 to display a message notifying the driver of abnormality occurrence, cause the speaker 15 to output the message by means of voice, alarm sound, or the like, or cause the vehicle external speaker 18 to output, to outer periphery of the vehicle, a message notifying that the driver's condition is abnormal by means of voice, alarm sound, or the like.

Subsequently in step S70, the controller 26 in the center management device 3 at the operation management center receives various information transmitted from the vehicle management device 2, specifically, the low-resolution video information on the driver image and the vehicle information at normal time, and causes the display device 21 to display the various information thus received. When the driver's condition is abnormal, the controller 26 in the center management device 3 receives various information transmitted from the vehicle management device 2-1 at abnormal time, specifically, the information indicating that the driver has difficulty in vehicle driving, the information on the determination result of the driver's condition, the high-resolution video information on the driver image (i.e. information having the resolution Rs), the biological information on the driver, the information on the current position of the vehicle, the vehicle information, and the like, and causes the display device 21 to display the various information thus received. In step S70, the operator at the operation management center visually recognizes the various information displayed on the display device 21 to monitor the driver's condition in each vehicle. The vehicle management device 2 may be referred to as a vehicle corresponding device.

In subsequent step S80, the operator at the operation management center determines whether the driver's condition is abnormal in accordance with each result of the monitoring. If the operator determines that the driver's condition is not abnormal (NO), the process flow returns to step S30 for repeated execution of the processing described above.

The process flow proceeds to step S90 if the operator determines that the driver's condition is abnormal (YES) in step S80. In this case, the operator at the operation management center determines whether the driver's condition requires emergency vehicle stop in accordance with the various information displayed on the display device 21, specifically, the information indicating that the driver has difficulty in vehicle driving, the information on the determination result of the driver's condition, the high-resolution video information on the driver image, the biological information on the driver, the information on the current position of the vehicle, the vehicle information, and the like.

The process flow proceeds to step S130 if the driver's condition does not require emergency vehicle stop (NO). In this case, the operator at the operation management center communicates with, in other words, contact by means of voice, the driver in the vehicle, by using the microphone 23 and the speaker 24 of the center management device 3, via the communicator 25, the mobile phone network 4, and the vehicle management device 2-1. The operator at the operation management center subsequently executes necessary treatment where appropriate while communicating with the driver in the vehicle.

The process flow proceeds to step S100 if the operator determines that the driver's condition requires emergency vehicle stop (YES) in step S90.

In this case, the operator at the operation management center operates the input device 22 of the center management device 3 to transmit a stop command signal for emergency vehicle stop to the vehicle management device 2-1 of the corresponding vehicle.

The process flow then proceeds to step S110, and the driver management ECU 10 in the vehicle management device 2-1 receives the stop command signal from the center management device 3 and transmits the stop command signal to the travel management ECU 17. The travel management ECU 17 accordingly drive-controls the accelerator, the brake, and the like of the vehicle via the vehicle controller 19 to stop the vehicle. The process flow then proceeds to step S130. The operator at the operation management center executes necessary treatment where appropriate while communicating with the driver in the vehicle (i.e. contacting by means of voice).

The vehicle management devices 2-1, 2-2, 2-3, 2-4 or the like thus configured in accordance with the present embodiment are each configured to transmit vehicle information and low-resolution video information on the driver image to the center management device 3 at normal time, and transmit vehicle information and high-resolution video information on the driver image to the center management device 3 when the driver's condition is abnormal. This configuration can reduce volume of data periodically transmitted from the vehicle management device 2-1, 2-2, 2-3, 2-4 or the like to the center management device 3 at normal time, for reduction in communication cost.

When the driver's condition is abnormal, the high-resolution video information on the driver image is transmitted to the center management device 3. Information sufficient for determination of the driver's condition is transmitted from the vehicle to the operation management center, so that the operator at the operation management center can visually recognize the driver image having high resolution and can quickly and accurately (i.e. on a real-time basis) determine that the driver's condition is abnormal. Particularly, when the driver's condition is abnormal, display of video information on the driver image according to the present embodiment is achieved by display on the display device 21 of the video information having the resolution Rs unified to the set value, in other words, clear driver images having the unified resolution. The operator at the operation management center can thus appropriately determine abnormality in driver's condition without variation in determination among the vehicles.

The driver management ECU 10 in each of the vehicle management devices 2-1, 2-2, 2-3, 2-4 or the like according to the embodiment described above is configured to transmit, to the center management device 3, video information having the resolution Rs unified to the set value when the driver's condition is abnormal, to prevent wasteful increase in communication time and communication cost.

The vehicle management device 2 according to the above embodiment is configured to transmit biological information on the driver to the center management device 3 when the driver's condition is abnormal. The operator at the operation management center thus checks the biological information on the driver to achieve more accurate determination that the driver's condition is abnormal.

According to the above embodiment, the center management device 3 is configured to transmit, to the vehicle management device 2, a stop command signal for vehicle stop in accordance with operation by the operator, and the vehicle management device 2 is configured to stop the vehicle upon receipt of the stop command signal from the center management device 3. This configuration achieves emergency vehicle stop according to external operation by the operator at the operation management center in a case where the driver is unconscious or the like to be unable to drive a vehicle, to achieve higher security of the vehicle.

### Second Embodiment

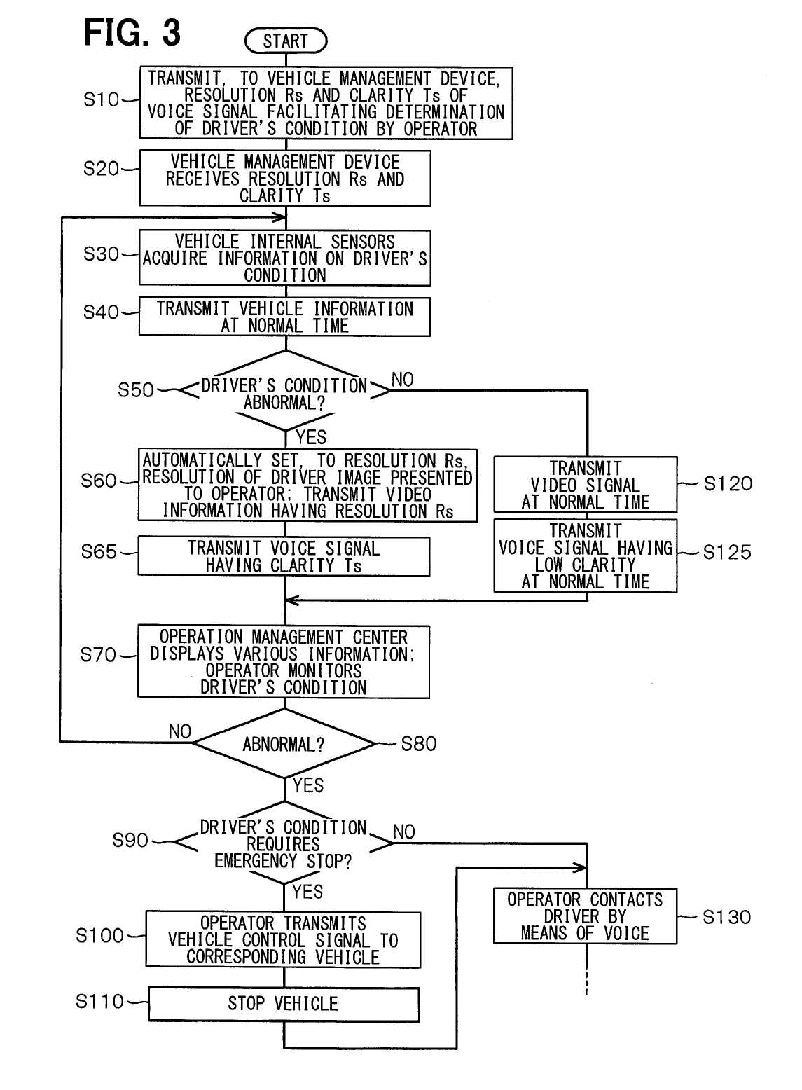

FIG. 3 relates to a second embodiment. A configuration identical to that according to the first embodiment is denoted by an identical reference sign. The second embodiment includes converting voice, sound, or the like generated by the driver in the vehicle into a voice signal by means of the microphone 7, and transmitting the voice signal (i.e. voice information) obtained by the conversion from the vehicle management device 2 to the center management device 3.

Specifically, in step S10 in FIG. 3, the operator at the operation management center operates the center management device 3 to transmit, to the vehicle management device 2-1, the resolution Rs of video information on the driver image to be received at abnormal time, and clarity Ts of the voice signal. The clarity Ts of the voice signal as additionally transmitted information corresponds to clarity of a voice signal necessary for output from the speaker 24 of voice facilitating determination of the driver's condition by the operator. The voice signal having the clarity Ts is larger in data volume than a voice signal having low clarity as information to be transmitted at normal time. The clarity Ts corresponds to clarity unified to a predetermined value.

In subsequent step S20, the vehicle management device 2-1 receives information on the resolution Rs and information on the clarity Ts of the voice signal from the center management device 3, and the driver management ECU 10 stores the resolution Rs and the clarity Ts thus received in the internal memory.

Processing from step S30 to step S60 and in step S120 is subsequently executed in a manner similar to that according to the first embodiment. The process flow proceeds from step S120 to step S125. The driver management ECU 10 in the vehicle management device 2-1 transmits, to the center management device 3, as information to be transmitted at normal time, the voice signal obtained by converting voice, sound, or the like generated by the driver in the vehicle by means of the microphone 7, specifically, the voice signal having low clarity or the voice signal having small data size in this case. In this case, the voice signal having low clarity is transmitted at communication speed of the communication line (i.e. the mobile phone network 4) being set to a low level.

The process flow proceeds from step S60 to step S65. The driver management ECU 10 in the vehicle management device 2 transmits, to the center management device 3, as information to be transmitted at abnormal time, the voice signal obtained by converting voice, sound, or the like generated by the driver in the vehicle by means of the microphone 7, specifically, the voice signal having the clarity Ts (i.e. high clarity unified to the predetermined value) or the voice signal having large data size in this case. In this case, the voice signal having high clarity is transmitted at communication speed of the communication line (i.e. the mobile phone network 4) being switched to a high level. The driver management ECU 10 thus configured functions as a voice output section.

Subsequently in step S70, the controller 26 in the center management device 3 at the operation management center is configured to cause the display device 21 to display the various information transmitted from the vehicle management device 2, and output the voice information transmitted from the vehicle management device 2 by means of voice or sound via the speaker 24. The controller 26 thus configured enables repeated output of the voice information transmitted from the vehicle management device 2 by means of voice or sound via the speaker 24 according to operation by the operator. Processing from step S80 to step S110 and in step S130 is to be executed in a manner substantially similar to that according to the first embodiment.

Configurations according to the second embodiment other than those described above are similar to corresponding configurations according to the first embodiment. The second embodiment thus achieves functional effect substantially same as that according to the first embodiment. The second embodiment particularly includes transmitting, to the center management device 3, the voice signal obtained by converting voice, sound, or the like generated by the driver in the vehicle by means of the microphone 7, so that the operator at the operation management center can determine the driver's condition more accurately. The second embodiment further includes transmitting, to the center management device 3, the voice signal having high clarity, specifically, the clarity Ts, for voice, sound, or the like generated by the driver in the vehicle when the driver's condition is abnormal. The operator at the operation management center can thus more accurately determine whether the driver's condition is abnormal.

The second embodiment still further provides the vehicle management devices 2-1, 2-2, 2-3, 2-4 or the like of the vehicles that are each configured to transmit, to the center management device 3, the voice signal having the clarity Ts having the unified predetermined value, for transmission to the center management device 3 of the voice signal having clarity when the driver's condition is abnormal. The operator at the operation management center can thus determine the drivers' conditions in the respective vehicles in accordance with the received voice signals having the clarity Ts more accurately without variation among the vehicles.

### Third Embodiment

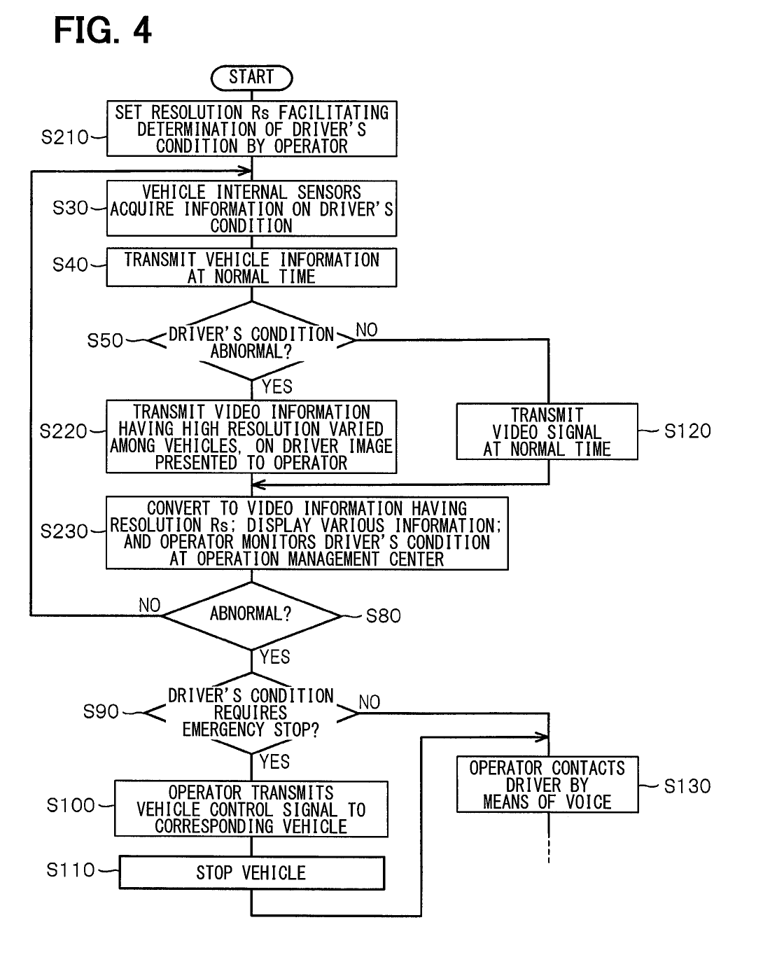

FIG. 4 relates to a third embodiment. A configuration identical to that according to the first embodiment is denoted by an identical reference sign. According to the third embodiment, the vehicle management devices 2-1, 2-2, 2-3, 2-4 or the like mounted on the plurality of vehicles each transmit, to the center management device 3, video information having high resolution possibly varied among the vehicle management devices 2-1, 2-2, 2-3, 2-4 or the like, for transmission of high-resolution video information on the driver images at abnormal time, and the center management device 3 converts the received video information to video information having the unified resolution Rs.

Specifically, in step S210 in FIG. 4, the center management device 3 (i.e. the operator) at the operation management center sets the resolution Rs of video information on the driver image to be received at abnormal time, and stores the resolution Rs thus set in an internal memory. Processing from step S30 to step S50 and in step S120 is subsequently executed in a manner similar to that according to the first embodiment.

If the driver's condition is abnormal (YES) in step S50, the process flow proceeds to step S220 and the driver management ECU 10 in each of the vehicle management devices 2-1, 2-2, 2-3, 2-4 or the like transmits, to the center management device 3, as information to be transmitted at abnormal time, the high-resolution video information on the driver image thus acquired, specifically, video information having high resolution possibly varied widely among the vehicles.

Subsequently in step S230, the controller 26 in the center management device 3 at the operation management center receives various information transmitted from the vehicle management device 2, specifically, the low-resolution video information on the driver image and the vehicle information at normal time, and causes the display device 21 to display the various information thus received. When the driver's condition is abnormal, the controller 26 in the center management device 3 receives various information transmitted from the vehicle management device 2-1 at abnormal time, specifically, the information indicating that the driver has difficulty in vehicle driving, the information on the determination result of the driver's condition, the high-resolution video information on the driver image (i.e. video information having high resolution possibly varied widely among the vehicles), the biological information on the driver, the information on the current position of the vehicle, the vehicle information, and the like, converts received video information having different high resolution to video information having the unified resolution Rs, and causes the display device 21 to display the various information thus received and the video information obtained by the conversion. Processing from step S80 to step S110 and in step S130 is configured to be executed in a manner similar to that according to the first embodiment.

Configurations according to the third embodiment other than those described above are similar to corresponding configurations according to the first embodiment. The third embodiment thus achieves functional effect substantially same as that according to the first embodiment.

Similarly to the third embodiment described above, the vehicle management devices 2-1, 2-2, 2-3, 2-4 or the like according to the second embodiment can alternatively be configured to transmit, to the center management device 3, video information having high resolution possibly varied, and the center management device 3 can be configured to convert the received video information to video information having the unified resolution Rs. In such a configuration, preferably, the vehicle management devices 2-1, 2-2, 2-3, 2-4 or the like are each configured to transmit, to the center management device 3, the voice signal having high clarity possibly varied, and the center management device 3 is configured to convert the received voice signal to voice information having the unified clarity Ts.

The embodiments described above include converting the video information having high resolution or the voice signals having high clarity to have the unified resolution Rs or the unified clarity Ts. The present invention is not limited to this configuration, but can alternatively include converting video information having low resolution or the voice signal having low clarity to have the set resolution or the set clarity.

The travel management ECU 17 in the vehicle management device 2 according to each of the above embodiments is configured to transmit, to the center management device 3, the high-resolution video information on the driver image, the voice signal having high clarity, or the like as information to be transmitted at abnormal time upon detection of abnormality in driver's condition. The present invention is not limited to this configuration. In a case where the operator at the operation management center monitoring the driver's condition at normal time requires detailed information for precise determination of whether the driver's condition is abnormal, the present invention can alternatively include transmitting, to the vehicle management device 2, command information commanding transmission to the center management device 3 of high-resolution video information on the driver image, a voice signal having high clarity, biological information on the driver, or the like, command information commanding turning on a lamp in the vehicle, command information commanding capturing a driver image with use of an infrared camera if such an infrared camera is mounted on the vehicle, or the like. In such a configuration, the driver management ECU 10 in the vehicle management device 2 is preferably configured to transmit, to the center management device 3, information commanded by any one of the command information thus received, specifically, the high-resolution video information on the driver image, the voice signal having high clarity, the biological information on the driver, or the like, or turn on the lamp in the vehicle in accordance with the corresponding command information, or cause any infrared camera mounted on the vehicle to capture a driver image.

The above embodiments each include transmission of the stop command signal from the operator (i.e. the center management device 3) at the operation management center to the vehicle management device 2 for vehicle stop. The present invention is not limited to this configuration. In a case where the driver management ECU 10 in the vehicle management device 2 determines abnormality of the driver and determines that it is better to stop the vehicle, the driver management ECU 10 can alternatively be configured to transmit the vehicle stop command signal to the travel management ECU 17 for vehicle stop.

The present disclosure has been described with reference to working examples, but the present disclosure should not be limited to these working examples or the configurations. The present disclosure can include various modification examples as well as modifications made within equivalent ranges. Furthermore, various combinations and modes, as well as other combinations and modes each obtained by adding only one or more elements to or removing any element from corresponding one of the various combinations and modes will fall within the scope and the ideological scope of the present disclosure.

It is noted that a flowchart or the processing of the flowchart in the present application includes multiple steps (also referred to as sections), each of which is represented, for instance, as S10. Further, each step can be divided into several sub-steps while several steps can be combined into a single step.

Controllers and methods described in the present disclosure may be implemented by a special purpose computer created by configuring a memory and a processor programmed to execute one or more particular functions embodied in computer programs. Alternatively, the control units, the controllers and the methods described in the present disclosure may be implemented by a special purpose computer created by configuring a processor provided by one or more special purpose hardware logic circuits. Further alternatively, the control units, the controllers and the methods described in the present disclosure may be implemented by one or more special purpose computers created by configuring a combination of a memory and a processor programmed to execute one or more particular functions and a processor provided by one or more hardware logic circuits. The computer programs may be stored, as instructions being executed by a computer, in a tangible non-transitory computer-readable medium.

While various embodiments, configurations, and aspects of vehicle operation management system according to the present disclosure have been exemplified, the embodiments, configurations, and aspects of the present disclosure are not limited to those described above. For example, embodiments, configurations, and aspects obtained from an appropriate combination of technical elements disclosed in different embodiments, configurations, and aspects are also included within the scope of the embodiments, configurations, and aspects of the present disclosure.

1. A vehicle operation management system comprising:
a plurality of vehicle management devices that are respectively mounted on vehicles; and
a center management device that is disposed at an operation management center and is communicable with the vehicle management devices,
wherein:
each of the vehicle management devices includes:
a vehicle information acquisition section that acquires vehicle information;
an image acquisition section that acquires a driver image;
a driver's condition determination section that determines whether a driver is in an abnormal condition; and
an information transmitter that transmits the vehicle information and low-resolution video information on the driver image to the center management device when the driver is in a normal condition, and transmits the vehicle information and high-resolution video information on the driver image to the center management device when the driver is in the abnormal condition;
the center management device includes:
an information receiver that receives the vehicle information and the low-resolution video information or the high-resolution video information on the driver image from each of the vehicle management devices;
an information display section that displays the vehicle information and the video information on the driver image; and
a speech section that allows an operator at the operation management center to communicate with a driver of the vehicles via the vehicle management devices; and
the information display section of the center management device displays video information having resolution unified to a set value for display of the video information on the driver image when the driver is in the abnormal condition.

2. The vehicle operation management system according to claim 1, wherein:
each of the vehicle management devices includes a voice information acquisition section that acquires voice information including voice or sound in the corresponding vehicle;
the information transmitter transmits voice information having a low clarity to the center management device when the driver is in the normal condition;
the information transmitter transmits voice information having a high clarity to the center management device when the driver is in the abnormal condition;
the information receiver of the center management device receives the voice information from the vehicle management devices;
the center management device includes a voice output section that outputs the voice information received as voice or sound; and
the voice output section outputs voice information having a clarity unified to a predetermined value as a voice or sound, in outputting the voice information received as voice or sound when the driver is in the abnormal condition.

3. The vehicle operation management system according to claim 1, wherein:
the information transmitter of each of the vehicle management devices transmits, to the center management device, the video information having a resolution unified to the set value when the driver is in the abnormal condition.

4. The vehicle operation management system according to claim 2, wherein:
the information transmitter of each of the vehicle management devices transmits, to the center management device, the voice information having a clarity unified to the predetermined value when the driver is in the abnormal condition.

5. The vehicle operation management system according to claim 1, wherein:
the center management device includes a stop command transmitter that transmits, to the vehicle management devices, a stop command signal for vehicle stop according to operation by the operator; and
each of the vehicle management devices includes a vehicle stop section that stops the vehicle upon receipt of the stop command signal from the center management device.

6. The vehicle operation management system according to claim 1, wherein:
a case when the driver is in the normal condition is a case where it is determined that a driver of a vehicle is not in a condition with a difficulty to drive the vehicle; and
a case when the driver is in the abnormal condition is a case where it is determined that the driver of the vehicle is in the condition to have the difficulty to drive the vehicle.

7. The vehicle operation management system according to claim 1, wherein:
a case when the driver is in the normal condition is a case where a driver of a vehicle cannot drive the vehicle; and
a case when the driver is in the abnormal condition is a case where the driver can drive the vehicle.

8. A vehicle operation management system comprising:
a vehicle apparatus that is mounted on a vehicle; and
a center apparatus that is disposed at an operation management center and is communicable with the vehicle apparatus,
wherein:
the vehicle apparatus includes an ECU that is configured to:
acquire vehicle information of the vehicle;
acquire an image of a driver as a driver image;
determine whether the driver is in an abnormal condition; and
transmit the vehicle information and low-resolution video information on the driver image to the center apparatus when it is determined that the driver is in a normal condition, and transmit the vehicle information and high-resolution video information on the driver image to the center apparatus when it is determined that the driver is in the abnormal condition;
the center apparatus includes a controller that is configured to:
receive the vehicle information and the low-resolution or high-resolution video information from the vehicle apparatus;
control a display device to display the vehicle information and the received video information; and
allow an operator at the operation management center to communicate with the driver of the vehicle via the vehicle apparatus; and
the display device displays video information having a predetermined resolution when the driver is in the abnormal condition.
